SEQUENCE LISTING (6 sequences)

SEQ ID NO: 1 - DNA, 20 bp, synthetic construct
  source 1..20: mol_type = other DNA
catggccttccgtgttccta

SEQ ID NO: 2 - DNA, 20 bp, synthetic construct
  source 1..20: mol_type = other DNA
catggccttccgtgttccta

SEQ ID NO: 3 - DNA, 18 bp, synthetic construct
  source 1..18: mol_type = other DNA
atgggcttagtgttctgg

SEQ ID NO: 4 - DNA, 18 bp, synthetic construct
  source 1..18: mol_type = other DNA
cctgttggctgagttgtg

SEQ ID NO: 5 - DNA, 20 bp, synthetic construct
  source 1..20: mol_type = other DNA
ggaggtgcatgtcctgaatc

SEQ ID NO: 6 - DNA, 20 bp, synthetic construct
  source 1..20: mol_type = other DNA
cagacttgtggtggatgtgg

(19) United States
(12) Patent Application Publication
(10) Pub. No.: US 2026/0224631 A1
(43) Pub. Date: Aug. 6, 2026

(54) COMPOSITION COMPRISING MIR-499-5P ACTIVATOR FOR PREVENTING OR TREATING DUCHENNE MUSCULAR DYSTROPHY

(71) Applicants: SAMSUNG LIFE PUBLIC WELFARE FOUNDATION, Seoul (KR); ENCELL CO., LTD., Seoul (KR)

(72) Inventors: Jong Wook CHANG, Seoul (KR); Hong Bae JEON, Seoul (KR); Sang Eon PARK, Seoul (KR); Shin Ji OH, Seoul (KR); Jang Bin JEONG, Seoul (KR)

(73) Assignees: SAMSUNG LIFE PUBLIC WELFARE FOUNDATION, Seoul (KR); ENCELL CO., LTD., Seoul (KR)

(21) Appl. No.: 18/723,656

(22) PCT Filed: Dec. 23, 2022
(86) PCT No.: PCT/KR2022/021174

§ 371 Date: Aug. 20, 2024

(30) Foreign Application Priority Data
Dec. 23, 2021 (KR) 10-2021-0186584

(51) Int. Cl. A61K 35/28; A61K 38/48; A61P 21/00; C12Q 1/6883

(52) U.S. Cl. CPC A61K 35/28; A61K 38/4886; A61P 21/00; C12Q 1/6883; C12Q 2600/158; C12Q 2600/178; C12Y 304/24007

(57) ABSTRACT

The present disclosure relates to a composition for preventing or treating Duchenne muscular dystrophy, including an miR-499-5p activator, wherein the miR-499-5p activator promotes expression of miR-499-5p by expressing MMP-1 in myotubes, and allows fibrous muscles to recover by down-regulating TGF-β receptor activation and smad2/3 phosphorylation. Thus, the composition can be used in the prevention or treatment of Duchenne muscular dystrophy.

### TECHNICAL FIELD

The present application claims priority to Korean Patent Application No. 10-2021-0186584, filed on Dec. 23, 2021, the disclosure of which is incorporated by reference in its entirety.

The present disclosure relates to a composition including an miR-499-5p activator for preventing or treating Duchenne muscular dystrophy.

### BACKGROUND ART

Duchenne muscular dystrophy (DMD) is an X-linked recessive disorder characterized by progressive loss of muscle mass and muscle function. It affects about 1 in 3,500 males and is caused by mutations in the dystrophin gene. It is characterized by progressive weakness in skeletal and cardiac muscle, and usually results in early death at around age 20. Currently, there is no effective treatment for DMD, and only treatments to slow the progression of the disease are available. Various therapeutic approaches for DMD have been attempted in preclinical and clinical studies, but no treatment with an effective clinical course has been reported yet to date. Furthermore, most of the treatments currently in development are merely gene therapies that target a single target against dystrophin. Since DMD involves multiple pathogenic mechanisms, such as myodegeneration, inflammation, and fibrosis, multi-targeted drug therapy may be more effective.

Recently, numerous clinical trials have shown that mesenchymal stem cells are a safe and beneficial treatment method for a variety of pathologies. Mesenchymal stem cells are mesodermal tissue-derived cells that can be obtained either from autologous or allogeneic sources, and that can differentiate into adipose, cartilage, and osteogenic lineages. Mesenchymal stem cells can also be engrafted into muscle tissue and differentiate into muscle cells. In addition, various proteins secreted by mesenchymal stem cells have therapeutic effects, and paracrine factors maintain homeostasis by regulating inflammation and immune responses at lesion sites, enabling multi-targeted therapy.

Therefore, there is a need to develop new therapies that can effectively treat DMD by controlling inflammation and immune responses at lesion sites.

### DISCLOSURE

### Technical Problem

An aspect is to provide a pharmaceutical composition for preventing or treating Duchenne muscular dystrophy (DMD), including an miR-499-5p activator.

Another aspect is to provide a cell therapy agent for preventing or treating DMD, including an miR-499-5p activator.

Another aspect is to provide a composition for diagnosing DMD, including an agent that measures an expression level of miR-499-5p.

Another aspect is to provide a kit for diagnosing DMD, including the composition.

Another aspect is to provide a method of providing information about diagnosis of DMD, including measuring an expression level of miR-499-5p in a sample of a subject.

### Technical Solution

An aspect provides a pharmaceutical composition for preventing or treating Duchenne muscular dystrophy (DMD), including an miR-499-5p activator. Another aspect provides a method of preventing or treating DMD, including administering an miR-499-5p activator to a subject in need thereof. An aspect provides therapeutic use of a pharmaceutical composition for preventing or treating DMD, including an miR-499-5p activator.

In the present specification, the term “microRNA (or miRNA or miR)” refers to a single-stranded RNA molecule having 21 nt to 25 nt in length that binds to the 3′UTR of mRNA and regulates gene expression in eukaryotes. In other words, the microRNA is involved in development, cell proliferation and death, fat metabolism, tumor formation, etc., by regulating expression of target proteins. In an embodiment, the miR-499-5p includes variants that are functional equivalents having changes that do not reduce activity of the miR-499-5p, the variants having one or more substations, insertions, deletions, and a combination thereof. In an embodiment, it was confirmed that the miR-499-5p promotes recovery of fibrous muscles by down-regulating a TGF-β receptor. In this regard, the miR-499-5p can bind specifically to the TGF-β receptor. The TGF-β receptor may be, for example, TGFβR1, TGFβR3, etc. That is, the miR-499-5p may have an anti-fibrotic effect by binding specifically to the TGF-β receptor.

In an embodiment, the miR-499-5p activator may be mesenchymal stem cells. The mesenchymal stem cells may be derived from umbilical cord, cord blood, bone marrow, placenta, adipose, etc. The umbilical cord may refer to a line that connects the mammalian fetus to the belly of a mother so that the mammalian fetus can grow in the placenta. In general, the umbilical cord may refer to a tissue composed of three vessels, i.e., two umbilical arteries and one umbilical vein, surrounded by Wharton's jelly. Therefore, in an embodiment, the mesenchymal stem cells may be derived from umbilical cord blood or cord blood, and specifically, Wharton's jelly-derived mesenchymal stem cells (WJ-MSCs). In an example, it was confirmed that, due to MMP-1 induced from mesenchymal stem cells in mdx mice, an expression level of miR-499-5p increased, exhibiting an anti-fibrotic effect. In this regard, in some embodiments, the mesenchymal stem cells may express MMP-1 or may be genetically engineered to overexpress MMP-1 as compared to parent cells.

In an example, it was confirmed that, by intravenously injecting WJ-MSCs at a specific cell number into mdx mice aged 3 to 5 months, therapeutic effects on skeletal muscle, such as reduction of intracellular creatine kinase, behavioral recovery, muscle regeneration, inhibition of apoptosis, reduction of fibrosis, etc, were exhibited. In other words, in mdx mice, the dose-dependent effect of WJ-MSCs and treatment of skeletal muscle by single intravenous administration rather than local administration were identified, and based on changes in the expression of miR-499-5p in skeletal muscle of the mdx mice, optimal dose and usage of WJ-MSCs for the treatment of DMD were identified. In this regard, the composition may include the mesenchymal stem cells in a cell count of 1×10² to 1×10⁸ cells. For example, the composition may include the mesenchymal stem cells in a cell count of 1×10² to 1×10⁸ cells, 1×10³ to 1×10⁷ cells, 5×10³ to 1×10⁷ cells, 5×10³ to 5×10⁶ cells, 1×10⁴ to 1×10⁶ cells, or 5×10⁴ to 1×10⁶ cells. Here, when the amount of the mesenchymal stem cells less than exceeds the ranges above, there is a problem in that reduction in creatine kinase levels, effects of increasing muscle strength, and effects of alleviating fibrosis cannot be sufficiently exhibited.

In an embodiment, the composition may be intravenously administered.

In general, DMD is the disease with the highest incidence among progressive muscular dystrophies, and is characterized by stiffening and enlargement of the gastrocnemius muscle in the early stages of the disease, followed by progressive degeneration and weakness of the skeleton and heart muscle of the entire body. Meanwhile, intramuscular (IM) injection is to administer a drug directly into the muscle, and has the advantage of fast absorption due to the abundance of blood vessels in the muscle. However, in the case of direct administration of the composition into the muscle, there is a problem in that the stem cells contained in the composition are limited in their migration from the site of administration to the surrounding area, requiring multiple administrations to different sites. On the other hand, intravenous (IV) injection is quick and clearly responsive because an injected drug passes through the heart and reaches the necessary tissue of the body within a minute or two. In particular, the stem cells can exhibit a homing effect, i.e., the ability to travel themselves to damaged areas, so that the stem cells can move to the skeletal muscle or diaphragm area where the disease appears and exhibit a therapeutic effect. In an example, as a result of confirming the distributions of WJ-MSCs in a normal control group where WJ-MSCs were intravenously administered and in mdx mice where WJ-MSCs were intravenously administered into the gastrocnemius muscle, it was confirmed that more cells remained in the gastrocnemius muscle of the mdx mice compared to the normal control group. That is, by injecting the composition including the miR-499-5p activator (e.g., WJ-MSCs) into the vein of a subject with DMD, the activator can migrate into the gastrocnemius muscle and exhibit a therapeutic effect, which is effective for the early treatment of DMD.

The pharmaceutical composition for preventing or treating DMD according to an aspect may have dosage forms, each according to methods known in the art, including a formulation for oral administration such as powders, granules, tablets, capsules, suspensions, emulsions, syrups, aerosols, and the like, an external preparation formulation, or a sterile injection solution formulation. For such dosage forms, suitable carriers, excipients, or diluents commonly used in the preparation of a pharmaceutical composition may be included.

For use as the carrier, excipient, and diluent, various compounds or mixtures including lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, or mineral oil may be used.

When forming into formulation, a commonly used diluent or excipient, such as a filler, an expander, a binder, a wetting agent, an disintegrant, a surfactant, and the like, may be used for preparation.

A solid preparation for oral administration may be prepared by mixing the beans extract with at least one excipient, such as starch, calcium carbonate, sucrose, lactose, gelatin, and the like. Also, in addition to a simple excipient, a lubricant, such as magnesium stearate and talc, may be used.

For use as a liquid formulation for oral administration, a suspension, an oral liquid, an emulsion, a syrup, and the like may be used. In addition to water and liquid paraffin, which are simple diluents commonly used, various excipients, such as a wetting agent, a sweetening agent, a flavoring agent, a preservative, and the like may be included.

For use as a formulation for parenteral administration, a sterile solution, a non-aqueous agent, a suspension, an emulsion, a freeze-dried agent, a suppository, and the like may be included. Examples of the non-aqueous agent and the suspension are propyleneglycol, polyethylene glycol, plant oil such as olive oil, and injectable ester such as ethyloleate. For use as a base agent for the suppository, witepsol, macrogol, tween 61, cacao butter, laurin butter, glycerol, gelatin, or the like may be used.

A preferable dosage of the pharmaceutical composition for preventing or treating DMD according to an embodiment may vary depending on a condition of a patient, a weight of a patient, severity of a disease, a drug form, an administration route, and an administration period, but may be appropriately selected by those skilled in the art. However, for a desirable effect, the dosage may be in a range of about 0.0001 mg/kg to about 2,000 mg/kg per day, preferably, about 0.001 mg/kg to about 2,000 mg/kg per day. The administration may be performed once a day or several times a day. However, the scope of the present disclosure is not limited by the dosage.

The pharmaceutical composition for preventing or treating DMD according to an aspect may be administered to mammals, such as rats, mice, livestock, humans, and the like, via various routes. All administration methods may be, for example, performed orally or rectally or by an intravenous, intramuscular, subcutaneous, intrauterine, or intracerebroventricular injection.

Another aspect provides a health functional food composition for preventing or ammeliorating DMD, the health functional food composition including an miRNA-499-5p activator as an active ingredient. Specific details of the miRNA-499-5p activator are as described above.

Regarding the health functional food for preventing or ameliorating DMD according to an aspect, when the compound is used as an additive to the health functional food, the compound may be added as it is or used in combination with other foods or food ingredients, appropriately according to methods known in the art. Here, a mixing amount of the active ingredient may be appropriately determined according to each purpose of use, such as prevention, health, or treatment.

The health functional food may be in any form of powders, granules, pills, tablets, or capsules, as well as a general food or beverage form.

Types of the general food are not particularly limited, and examples of the general food to which the substance can be added include meat, sausage, bread, chocolate, candy, snacks, confectionery, pizza, ramen, other noodles, gum, dairy products including ice cream, various soups, beverage, tea, drinks, alcoholic beverages, vitamin complex, and the like, and may include all foods in the ordinary sense.

In general, when preparing a food or a beverage, the compound may be added in an amount of 15 parts by weight or less, preferably, 10 parts by weight or less, based on 100 parts by weight of the raw material. However, in the case of long-term intake for the purpose of health and hygiene or health control, the amount may be less than the ranges above. Also, since there is no problem in terms of safety in that fractions from natural products are used, the amount greater than the ranges above may be used.

In the health functional food according to an aspect, a beverage may contain, as additive ingredients, various flavoring agents or native carbohydrates as in general beverages. Examples of the native carbohydrate may include monosaccharides, such as glucose and fructose, disaccharides, such as maltose and sucrose, polysaccharides, such as dextrin and cyclodextrin, and sugar alcohols, such as xylitol, sorbitol, and erythriotol. For use as the sweetening agent, a natural sweetening agent, such as thaumatin and a stevia extract, a synthetic sweetening agent, such as saccharin, aspartame, and the like may be used. A proportion of the native carbohydrate in the beverage may be in a range of about 0.01 g to about 0.04 g, preferably, about 0.02 g to about 0.03 g, per 100 mL of the beverage according to the present disclosure.

Furthermore, the health functional food for preventing or ameliorating DMD according to an aspect may include various nutrients, vitamins, electrolytes, flavors, coloring agents, pectic acid and a salt thereof, alginic acid and a salt thereof, organic acids, protective colloidal thickeners, pH adjusters, stabilizers, preservatives, glycerin, alcohol, carbonizing agent used in carbonated beverages. Moreover, the composition for sleep improvement of the present disclosure may include fruit pulps for preparing a natural fruit juice, a fruit juice beverage, and a vegetable juice. Such components may be used independently or in combination. A ratio of these additives is not limited, but is generally selected from the range of about 0.01 parts by weight to about 0.1 parts by weight based on 100 parts by weight of the health functional food of the present disclosure.

Another aspect provides a cell therapy agent for preventing or treating DMD, the cell therapy agent including an miR-499-5p activator. Specific details of the miR-499-5p activator are as described above.

In the present specification, the term “cell therapy agent” refers to a drug for the purpose of treatment, diagnosis, and prevention through a series of actions that change biological characteristics of cells by in vitro proliferation, selection, or various methods using living autologous, allogenic, or xenogenic cells to restore functions of cells and tissues.

As described above, the miR-499-5p activator according to an aspect promotes the expression of miR-499-5p by expressing MMP-1 in myotubular cells, and restores fibrotic muscle by down-regulating activation of TGF-β receptors and phosphorylation of smad2/3, and thus may be used for the prevention or treatment of DMD.

Another aspect provides a composition for diagnosing DMD, the composition including an agent capable of specifically detecting an expression level of miR-499-5p. Specific details of the miR-499-5p are as described above.

In the present specification term “agent capable of specifically detecting an expression level of miRNA” refers to a substance that can be used to specifically identify and detect the miRNA in a sample of a subject. The agent may be, for example, a primer, a probe, or an antisense nucleic acid.

Another aspect provides a kit for diagnosing DMD, the kit including the composition. Specific details of the composition are as described above.

The kit may include a primer, probe, or antisense nucleic acid, which cap specifically detect the expression level of miR-499-5p, and may further include one or more other component compositions, solutions, or devices suitable for the assay. In an embodiment, the kit may be an RT-PCR kit, a microarray chip kit, or a protein kit.

Another aspect provides a method of providing information about diagnosis of DMD, the method including measuring an expression level of miR-499-5p in a sample of a subject. Specific details of the miR-499-5p are as described above. In an embodiment, the method may further include determining that the subject has DMD when the expression level of miR-499-5p is decreased as compared to the control group.

Here, the subject refers to a patient for whom DMD is to be diagnosed. The subject may be a vertebrate, mammal, amphibian, reptile, bird, etc., or may be a mammal such as a human (Homo sapiens), a Korean.

Such a biological sample may include samples such as tissue, tumor tissue, lung tumor tissue, cells, whole blood, serum, plasma, saliva, sputum, cerebrospinal fluid, or urine, isolated from an individual.

The measuring of the expression level of miR-499-p may be performed by Western blotting, reverse-transcription polymerase chain reaction (RT-PCR), competitive RT-PCR, real-time RT-PCR, RNase protection assay (RPA), Northern blotting, DNA chip, etc.

Another aspect provides a method of screening a therapeutic agent for DMD, including: screening a test substance having an increased expression level of miR-499-5p compared to an untreated control group as a therapeutic agent for DMD. Another aspect provides a method of screening a therapeutic agent for DMD, including: contacting a test substance to muscle cells having a decreased expression level of miR-499-5p compared to a normal control group; measuring a level of fibronectin or p-smad2/3 in the muscle cells; and screening, as a therapeutic agent for DMD, the test substance that decreases a level of fibronectin or p-smad2/3 compared to a control group not treated with the test substance. Specific details of the miR-499-5p are as described above. Here, the test substance, such as a drug candidate, a test compound, or a test composition, may include a small molecule drug, an antibody, an antisense nucleotide, a short interfering RNA, a short hairpin RNA, a nucleic acid, a protein, a peptide, other extracts, or natural products. The contacting may be performed in vitro.

### Advantageous Effects

In the composition according to an aspect, the miR-499-5p activator promotes the expression of miR-499-5p by expressing MMP-1 in myotubular cells, and restores fibrotic muscle by down-regulating activation of TGF-β receptors and phosphorylation of smad2/3, and thus the composition may be used for the prevention or treatment of DMD.

### DESCRIPTION OF DRAWINGS

FIG. 1A shows the results of measuring mouse muscle strength after intravenous injection of WJ-MSCs into an mdx mouse model.

FIG. 1B shows the results of measuring a level of creatine kinase in serum after intravenous injection of WJ-MSCs into mdx mice.

FIG. 1C shows the results confirming a fibrotic area, a myosin heavy chain (MHC), and an expression level of annexin V, after intravenous injection of WJ-MSCs into mdx mice.

FIG. 1D shows the results confirming a fibrotic area, a myosin heavy chain (MHC), and an expression level of annexin V, obtained by using Image J, after intravenous injection of WJ-MSCs into mdx mice.

FIG. 2A shows a heatmap of commonly expressed miRNAs in humans, mdx mice, and healthy mice.

FIG. 2B shows relative miRNA-499 expression before and after intravenous injection of WJ-MSCs into mdx mice.

FIG. 2C shows the results confirming expression levels of miR-499-5p due to MMP-1 protein.

FIG. 3A shows the results of confirming relative expression levels of TGFβR1 before and after intravenous injection of WJ-MSCs into mdx mice.

FIG. 3B shows the results confirming relative expression levels of TGFβR3 before and after intravenous injection of WJ-MSCs into mdx mice.

FIGS. 4A and 4B show the results confirming the anti-fibrotic effect on skeletal muscle after intravenous injection of WJ-MSCs into mdx mice.

FIGS. 4C and 4D show the results confirming the anti-fibrotic effect on diaphragm after intravenous injection of WJ-MSCs into mdx mice.

FIGS. 5A and 5B show the results of measuring Smad2/3 phosphorylation levels after intravenous injection of WJ-MSCs into mdx mice.

FIG. 6A shows the results of quantifying DNA in organs after intravenous injection of WJ-MSCs into a normal control group and mdx mice.

FIGS. 6B and 6C show the results of measuring fibrosis over time after intravenous injection of WJ-MSCs into mdx mice.

FIG. 7A shows the results of transfection with miR-499-5p or treatment with a TGF-β inhibitor after inducing oxidative stress in mouse myotubes.

FIG. 7B shows results confirming collagen accumulation in mouse myotubes transfected with miR-499-5p after inducing oxidative stress in mouse myotubes.

FIG. 7C shows results confirming collagen I accumulation in mouse myotubes transfected with miR-499-5p after inducing oxidative stress in mouse myotubes.

FIG. 7D shows the results confirming expression of myosin heavy chain, fibronectin, and p-smad2/3 by inducing oxidative stress in mouse myotubes followed by transfection with miR-499-5p or treatment with a TGF-β inhibitor.

FIG. 8 shows the anti-fibrotic effect of miR-499-5p in skeletal muscle of DMD.

### BEST MODE

Hereinafter, preferable Examples are presented to help understanding of the present disclosure. However, Examples below are only presented for easier understanding of the present disclosure, and the contents of the present disclosure are not limited by the following examples.

### EXAMPLES

### Example 1. Confirmation of Therapeutic Effect of Wharton's Jelly-Derived Mesenchymal Stem Cell (WJ-MCS) on Muscular Dystrophy

### 1-1. Measurement of Grip Strength

To confirm the therapeutic effect of mesenchymal stem cells on muscular dystrophy, the muscle strength of the forelimbs and hindlimbs of mice was measured. First, WJ-MSCs were isolated according to a known method, and then cultured at the Good Manufacturing Practice Facility of Samsung Seoul Hospital according to the standard operating procedures. Afterwards, the WJ-MSCs were intravenously injected into the tail of 3 to 5 months-old C57BL/10ScSn-Dmdmdx/J (mdx) mice having DMD (available from the Jackson Laboratory, USA) at various doses (5×10³ (Dose 1), 1×10⁴ (Dose 2), 5×10⁴ (Dose 3), 1×10⁵ (Dose 4), or 5×10⁵ (Dose 5)). Aggregated cells can cause intravascular embolism, and thus the WJ-MSCs were mixed with 100 μL of PBS to prepare a suspension before injection, and then injected as slowly as possible. Next, by using a grip strength meter (BIO GS3, BIOSEB, Vitrolles, France), the muscle strength of the forelimbs and/or hindlimbs of the mice was measured. As a control group, C57BL/10ScSnJ mice (available from the Jackson Laboratory, USA) were used.

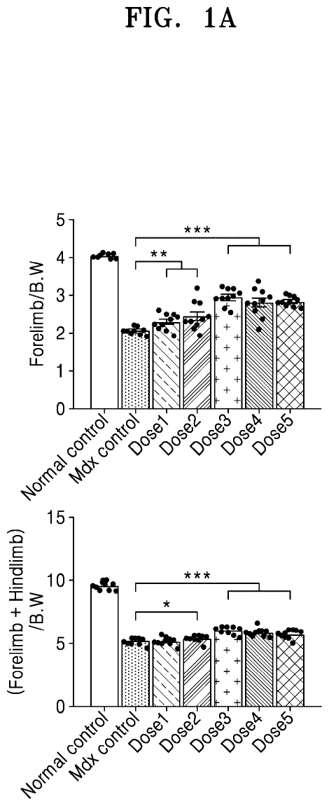

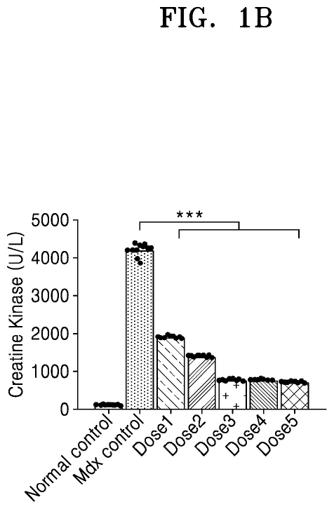

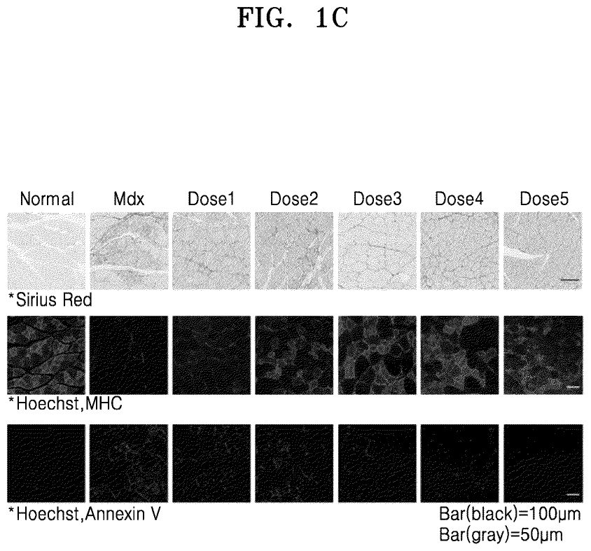

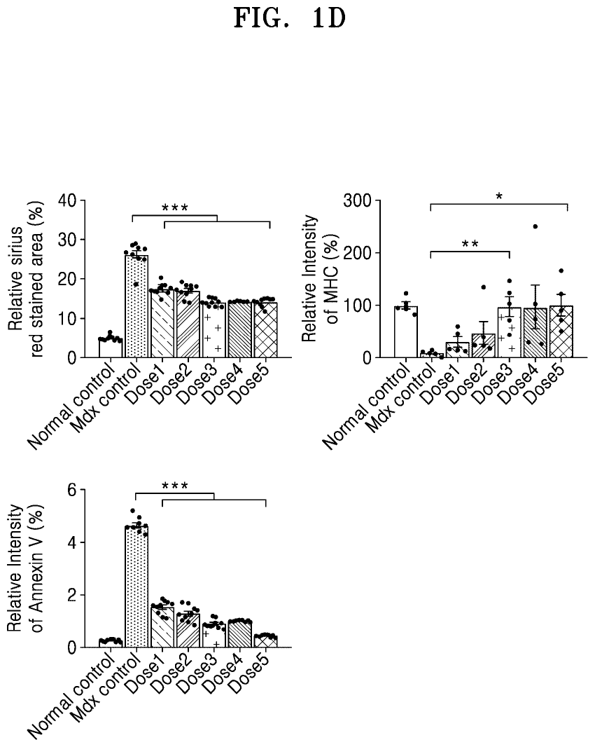

FIG. 1A shows the results of measuring the mouse muscle strength after intravenous injection of the WJ-MSC into an mdx mouse model.

Consequently, as shown in FIG. 1A, it was confirmed that the muscle strength of the (left) forelimb and (right) hindlimb of the mdx mice administered with the WJ-MSCs increased as compared to the control group not administered with the WJ-MSCs. In particular, the muscle strength increased in a concentration-dependent manner at Doses 1 to 3, and tended to decrease at Dose 4 and higher. In other words, the effect of increasing muscle strength can be achieved by administering the WJ-MSCs at a specific dose.

### 1-2. Measurement of Creatine Kinase (CK) Levels in Serum

To determine the dose-dependent effect of symptom alleviation by mesenchymal stem cells, the levels of creatine kinase (CK) in mouse serum was measured. Specifically, the mouse blood was collected by inducing retro-orbital plexus bleeding in the mdx mice administered with the WJ-MSCs in Example 1-1, incubated at 22 to 24° C. for at least 1 hour, and centrifuged at 15,000×g for 10 minutes, so as to collect an upper layer of clear serum. Next, the CK activity in serum was measured by using a creatine kinase activity assay kit (colorimetric assay).

FIG. 1B shows the results of measuring the CK level in serum after intravenous injection of the WJ-MSCs into the mdx mice.

Consequently, as shown in FIG. 1B, the CK level was significantly decreased in the mdx mice administered with the WJ-MSCs as compared to the control group not administered with the WJ-MSCs. In particular, administration of Doses 3 to 5 showed a significant reduction in the CK levels compared to administration of lower doses.

### 1-3. Confirmation of Intramuscular Fibrosis Level, Regeneration of Muscle Cells, and Anti-Apoptosis Effect

The degree of fibrosis, regeneration of muscle cells, and anti-apoptosis effects in the muscles of the mdx mice by the administration of mesenchymal stem cells were determined. In detail, the mdx mice administered with the WJ-MSCs in Example 1-1 were euthanized. After removing the mouse skin, the gastrocnemius muscle and diaphragm were removed and fixed with 4% paraformaldehyde for 24 hours. Next, the muscles were embedded in paraffin, cut into 4 μm sections, and stained with Sirius red to observe muscle fibrosis. The regeneration of muscle cells was detected by culturing the fixed gastrocnemius muscle sections with myosin heavy chain (MHC) antibodies (#MAB4470, diluted 1/1000; R&D Systems, MN, USA) at 4° C. for 18 hours, and culturing again with secondary antibodies, Alexa Fluor® 488 AffiniPure Goat Anti-mouse IgG (H+L) (A10680, Thermo Fisher Scientific, Waltham, MA, USA). Afterwards, the gastrocnemius muscle sections were counter-stained with Hoechst 33342 (H1339, Thermo Fisher Scientific).

FIG. 1C shows the results confirming a fibrotic area, a myosin heavy chain (MHC), and an expression level of annexin V, after intravenous injection of the WJ-MSCs into the mdx mice.

FIG. 1D shows the results confirming a fibrotic area, a myosin heavy chain (MHC), and an expression level of annexin V, obtained by using Image J, after intravenous injection of the WJ-MSCs into the mdx mice.

Consequently, as shown in FIGS. 1C and 1D, the Sirius red staining areas were significantly reduced in the mdx mice administered with the WJ-MSCs as compared to the control group not administered with the WJ-MSCs, and fibrosis was alleviated by administration of the WJ-MSCs at Dose 3. In addition, as compared to the control group not administered with the WJ-MSCs, the intensity of the MHC was significantly increased in the mdx mice administered with the WJ-MSCs, and the intensity of the MHC was remarkably increased by the administration of Dose 3, demonstrating the regeneration effect of muscle cells. In addition, as compared to the control group not administered with the WJ-MSCs, the expression level of annexin V decreased in the mdx mice administered with the WJ-MSCs in a concentration-dependent manner when Doses 1 to 3 of the WJ-MSCs were administered, but the expression level of annexin V was found to increase by administration of the WJ-MSCs at Dose 4. Referring to the results above, it was confirmed that Dose 3 of the WJ-MSCs is the smallest dose to exhibit the therapeutic effect. In other words, since the effects of fibrosis alleviation, muscle regeneration, and anti-apoptosis did not significantly increase at high doses, Dose 3 was determined as the optimal dose for treating muscular dystrophy.

### Example 2. Sequencing and Analysis of miRNA Expressed in Muscular Dystrophy

2-1. Identification of Differentially Expressed miRNA in Muscular Dystrophy

To identify differentially expressed miRNAs in muscular dystrophy, miRNA sequencing and data analysis were performed. In detail, total RNAs were extracted from the gastrocnemius skeletal muscle of the mouse of Example 1-3 by using TRIzol Reagent (Invitrogen, Waltham, MA, USA). Next, Agilent 2100 Bioanalyzer and RNA 6000 Pico Chips (Agilent Technologies, Amsterdam, The Netherlands) were used to evaluate the RNA quality, and NanoDrop 2000 spectrophotometer (Thermo Fisher Scientific) was used to measure the RNA concentration. Construction of miRNA library was performed by using NEBNext Multiplex Small RNA Library Prep kit (New England BioLabs, Inc., Ipswich, MA, USA), and single-end sequencing of 75 bp reads was performed by using a NextSeq500 system (Illumina, San Diego, CA, USA) to produce high-throughput sequences. The sequence reads were mapped by using Bowtie 2, and the mature miRNA sequences were used for reference. The read counts mapped to the mature miRNA sequences were extracted from an alignment file by using a bioconductor using bedtools (v2.25.0) and the R statistical programming language (version: 3.2.2; R development Core Team, 2011). Comparison between samples were made by using a quantile normalization method, and clustering heatmap was performed by using MeV (version: 4.9.0).

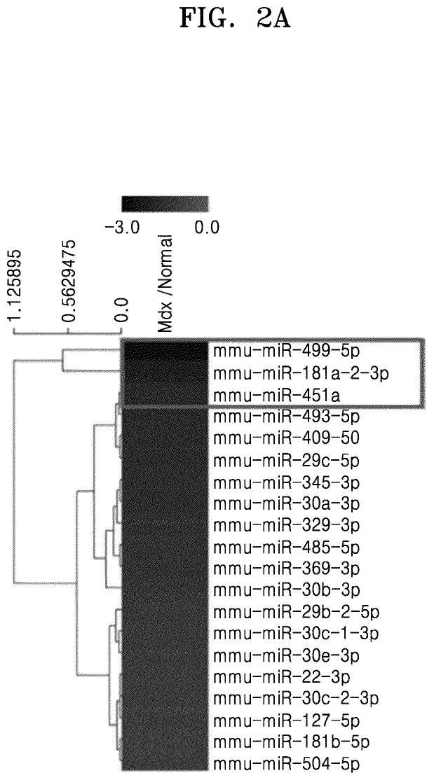

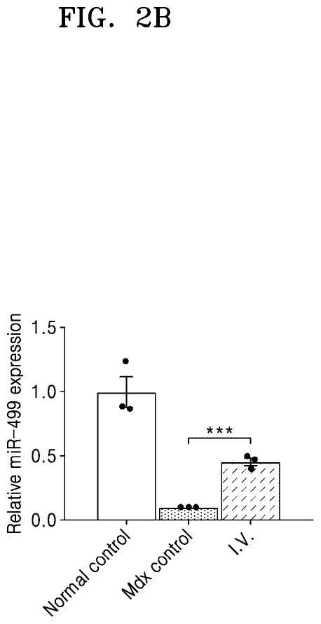

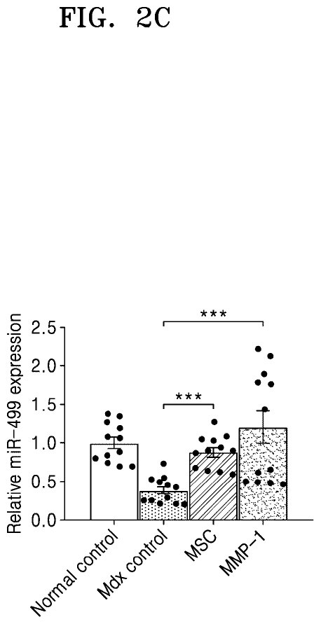

FIG. 2A shows a heatmap of commonly expressed miRNAs in humans, mdx mice, and healthy mice.

Consequently, as shown in FIG. 2A, several miRNAs were down-regulated in mice having muscular dystrophy as compared to healthy mice, and among them, the expression of miR-499-5p was found to decrease the most.

2-2. Confirmation of Expression Level of miR-499-5p in Muscular Dystrophy

To confirm the miRNA sequencing results obtained in Example 2-1, the expression level of miR-499-5p in the mice having muscular dystrophy was analyzed. In detail, miRNAs were isolated from the gastrocnemius skeletal muscle of the mouse of Example 1-3 by using the mirVana™ miRNA isolation kit (Invitrogen). Afterwards, the miRNA concentration was measured by using the Qubit microRNA assay kit and the Qubit 4 fluorometer (Invitrogen). The isolated miRNAs were reverse-transcribed into cDNAs at a concentration of 5 ng/μL by using the TaqMan™ Advanced miRNA cDNA synthesis kit (Applied Biosystems, Waltham, MA, USA). Afterwards, qPCR was performed on the QuantStudio™ 6 Flex real-time PCR system (Applied Biosystems) by using TaqMan™ Fast Advanced Master Mix (Applied Biosystems) in a fast cycling mode. The level of hsa-miR-499-5p (analysis ID: 478139_mir) was measured by Taqman™ Advanced miRNA analysis (Invitrogen). All reactions were performed in triplicate, and comparative quantification of each target gene was performed based on cycle threshold (CT) values normalized to miR-26a-5p according to a DDCT method proposed by Livak and Schmittgen.

FIG. 2B shows relative miRNA-499 expression before and after intravenous injection of the WJ-MSCs into mdx mice.

Consequently, as shown in FIG. 2B, the expression of miR-499-5p was relatively reduced in the mdx mice as compared to the normal control group, but when the WJ-MSCs were injected into mdx mice, the expression of miR-499-5p was found to significantly increase. That is, the miR-499-5p is miRNA that is down-regulated in DMD, and the expression level thereof is found to increase by intravenous injection into mesenchymal stem cells.

2-3. Confirmation of Expression Level of miR-499-5p Due to MMP-1 Protein

In previous studies, it has been confirmed that the WJ-MSCs alleviated the fibrosis of skeletal muscle through MMP-1 in the mdx mice and in vitro fibrosis models. Therefore, based on the results above, it was confirmed whether the expression level of miR-499-5p was increased by the MMP-1 protein. In detail, 20 ng of human recombinant MMP-1 was injected into mdx mice, and the mice were sacrificed on the 7th day. Afterwards, in the same manner as in Example 2-2, the expression level of miR-499-5p was confirmed.

FIG. 2C shows the results confirming the expression level of miR-499-5p due to the MMP-1 protein.

Consequently, as shown in FIG. 2C, the expression level of miR-499-5p decreased in the mdx mice as compared to the control group, and but due to the MMP-1 protein, the expression level of miR-499-5p in the mdx mice was found to recover to the level equivalent to or higher than the expression level of miR-499-5p in the control group. That is, the WJ-MSCs induce MMP-1 to increase the expression level of miR-499-5p in muscular dystrophy, exhibiting the anti-fibrotic effect.

### Example 3. Identification of Target Receptor for miRNA-499-5p

It was confirmed which mechanism is involved to cause an increase in miR-499-5p by mesenchymal stem cells in the treatment of muscular dystrophy. First, a target receptor of miRNA-499-5p was predicted by using a bioinformatics method (TargetScan). As a result, highly assisted miR-499-5p target sites were detected in TGFβR1 and TGFβR3 sites in the mice. Accordingly, it was confirmed whether there was a direct binding to miRNA-499-5p by measuring the expression levels of TGFβR1 and TGFβR3. In detail, total RNAs were extracted from the skeletal muscle gastrocnemius skeletal muscle of the mice of Example 1-3 by using a TRIzol Reagent. Afterwards, the RNAs were reverse-transcribed into cDNAs by using a SuperScript IV reverse transcriptase (Invitrogen). PCR amplification was performed by using primers listed in Table 1 and 2×Power SYBR Green Master Mix (Applied Biosystems) by initial denaturation at 95° C. for 10 minutes, followed by 40 cycles of denaturation at 95° C. for 15 seconds and 55° C. for 55 seconds.

TABLE 1

| | SEQ ID | | |
|---|---|---|---|
| Protein | NO: | Primer | |
| GAPDH | 1 | Foward | 5′-CATGGCCTTCCGTGTTCCTA-3′ |
| | 2 | Reverse | 5′-CATGGCCTTCCGTGTTCCTA-3′ |
| mouse | 3 | Foward | 5′-ATGGGCTTAGTGTTCTGG-3′ |
| TGFβR1 | 4 | Reverse | 5′-CCTGTTGGCTGAGTTGTG-3′ |
| mouse | 5 | Foward | 5′-GGAGGTGCATGTCCTGAATC-3′ |
| TGFβR3 | 6 | Reverse | 5′-CAGACTTGTGGTGGATGTGG-3′ |

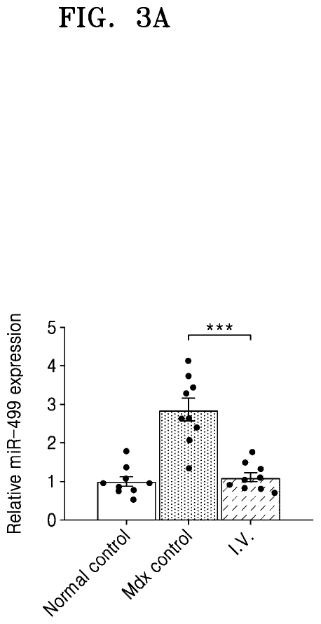

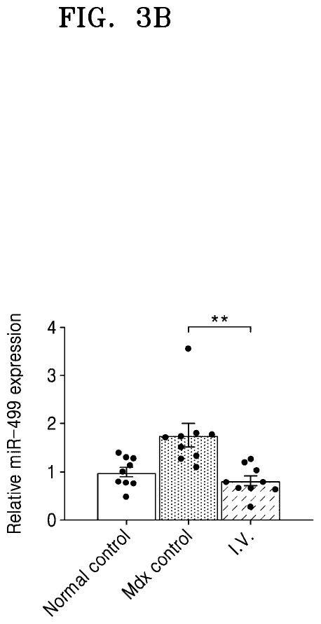

FIG. 3A shows the results of confirming relative expression levels of TGFβR1 before and after intravenous injection of the WJ-MSC into mdx mice.

FIG. 3B shows the results of confirming relative expression levels of TGFβR3 before and after intravenous injection of the WJ-MSC into mdx mice.

Consequently, as shown in FIGS. 3A and 3B, the expression of TGFβR1 and TGFβR3 significantly increased in the mdx mice as compared to the control group, but after injection of the WJ-MSCs, the expression of TGFβR1 and TGFβR3 was found to decrease to a level similar to that of the normal control group.

That is, the miR-499-5p was confirmed to target TGFβR1 and TGFβR3, which are key factors in fibrosis.

### Example 4. Confirmation of Therapeutic Effect on Muscular Dystrophy

### 4-1. Confirmation of Anti-Fibrotic Effect in Skeletal Muscle and Diaphragm

The anti-fibrotic effect of the mesenchymal stem cells on muscular dystrophy was confirmed. In detail, in the same manner as in Example 1-3, the skeletal muscle and diaphragm of the mdx mice (injected at Dose 3) obtained in Example 1-3 were stained with H&E and Sirius red to observe muscle fibrosis. In addition, the immunohistochemistry was performed in the same manner as in Example 1-3, except that fibronectin antibodies (ab2413, at 1/200 dilution, Abcam) primary antibodies and Alexa Fluor® 594 AffiniPure Goat Anti-rabbit IgG (H+L) (A11037, Thermo Fisher Scientific) as secondary antibodies.

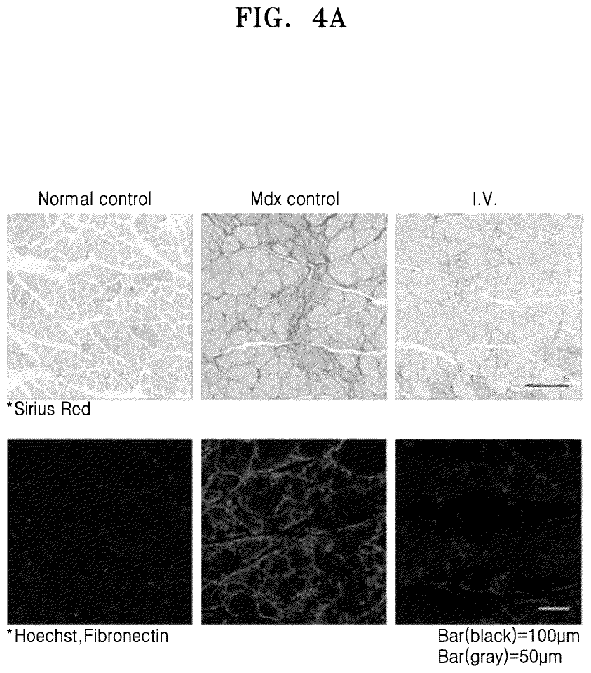

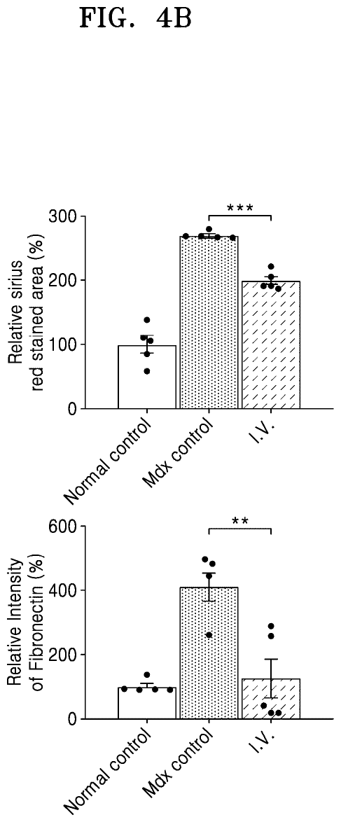

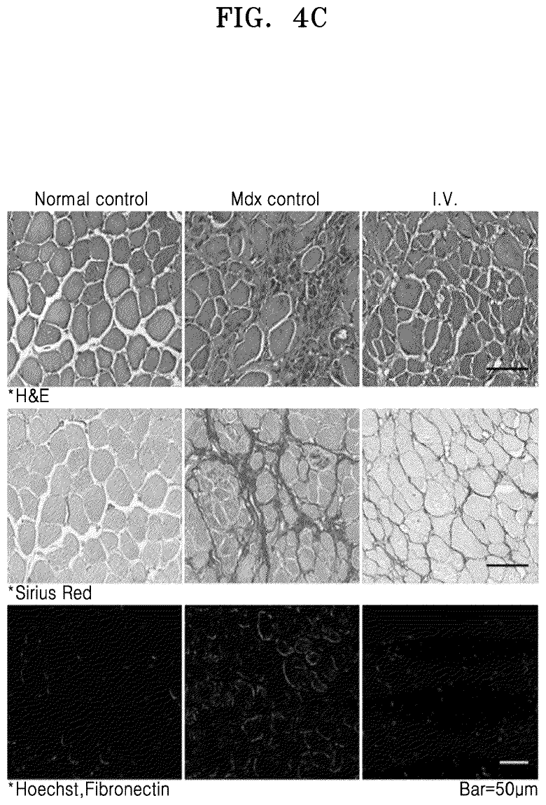

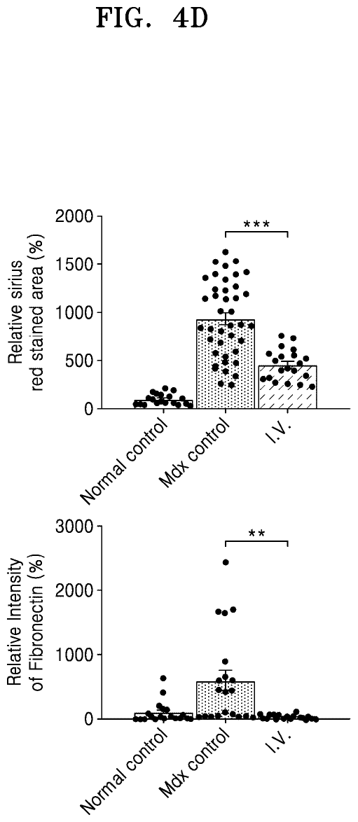

FIGS. 4A and 4B show the results confirming the anti-fibrotic effect on skeletal muscle after intravenous injection of WJ-MSC into mdx mice.

FIGS. 4C and 4D show the results confirming the anti-fibrotic effect on diaphragm after intravenous injection of WJ-MSC into mdx mice.

Consequently, as shown in FIG. 4A to 4D, the detection intensity for Sirius red staining areas and fibronectin was relatively increased as compared to the control group, but was found to significantly reduce by the injection of the WJ-MSCs. In particular, the intensity of fibronectin detection in the muscle cells and diaphragm tissue was found to reduce to a level similar to or lower than that of the normal control group. In other words, the mesenchymal stem cells were effective in the prevention or treatment of muscular dystrophy by reducing muscle fibrosis.

### 4-2. Confirmation of Anti-Fibrotic Effect by TGF-β Signaling

To confirm the anti-fibrotic effect by the TGF-β signaling, the level of Smad2/3 phosphorylation was measured according to Western blotting. Specifically, mouse myoblast cells, C2C12 (ACTC CRL-1772), were cultured under conditions of 5% CO₂ and 37° C. in a Dulbecco's modified Eagle's medium (DMEM, Biowest S.A.S, Nuaille, France) supplemented with 10% bovine serum (Gibco BRL, MA, USA), 100 U/mL penicillin, and 100 μg/mL streptomycin (Gibco BRL). Afterwards, the culture medium was replaced with a differentiation medium supplemented with 5% horse serum (Gibco BRL) for 5 days to differentiate the cells into myotubes. The myotubes were scraped from the culture dish and dissolved in an ice-cold radioimmunoprecipitation buffer solution (9.8 mol/L UREA, 4% CHAPS, 130 mmol/L dithiothreitol, 40 mmol/L tris HCl, 0, 1% sodium dodecyl sulfate, 1 mmol/L EDT, and protease/phosphatase inhibitor cocktail) to extract total proteins. Afterwards, the proteins were quantified according to Bradford assay (Bio-Rad Laboratories, Hercules, CA, USA). The proteins of equal amounts were separated by sodium dodecyl sulfate-polyacrylamide gel electrophoresis and transferred to a polyvinylidene difluoride film (Bio-Rad Laboratories). After blocking with 5% skim milk, the cells were cultured overnight at 4° C. with fibronectin (ab2413, 1/10,000 dilution, Abcam), p-smad2/3 (8828S, 1/1000 dilution, Cell Signaling Technology, Danvers, MA, USA), and smad2/3 (3102S, 1/1000 dilution, Cell Signaling Technology) as a primary antibody. Afterwards, the membrane was washed three times with TBST, and incubated at room temperature for 1 hour with a goat anti-rabbit IgG HRP-conjugated antibody (GTX213110-01, 1/10,000 dilution; GeneTex, Irvine, CA, USA) or a goat anti-mouse IgG HRP-conjugated antibody (GTX213111-01, 1/10,000 dilution; GeneTex) as a secondary antibody.

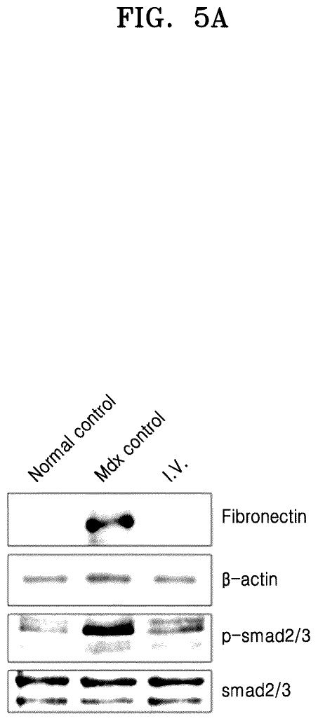

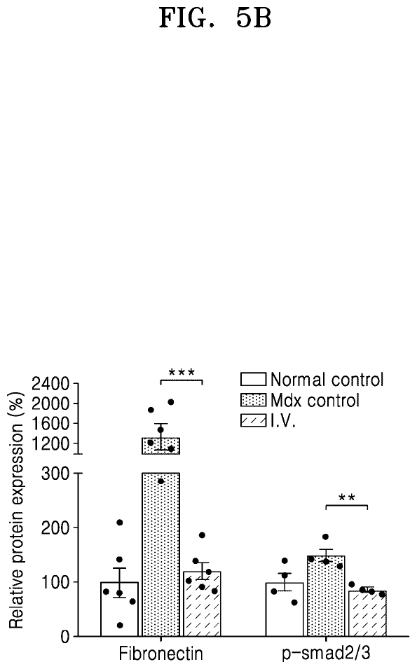

FIGS. 5A and 5B show the results of measuring Smad2/3 phosphorylation levels after intravenous injection of the WJ-MSC into mdx mice.

Consequently, as shown in FIGS. 5A and 5B, the expression of fibronectin and p-smad2/3 significantly increased in the mdx mice as compared to the control group, but was found to decrease to a level similar to that of the normal control group in the presence of the WJ-MSCs. In other words, the Smad2/3 phosphorylation shows a similar pattern to the expression of fibronectin, and thus the fibrosis can be inhibited by activating the TGF-β signaling pathway.

### 4-3. Confirmation of Intra-Tissue Distribution of WJ-MSC

The distribution of mesenchymal stem cells in tissue was to be confirmed. Specifically, the WJ-MSCs present in the gastrocnemius muscle, heart, liver, lung, and spleen of the mdx mice intravenously administered with the WJ-MSCs in Example 1-1 were quantified. PCR was performed in 20 μL of reactants including 10 μL of 2×Power SYBR™ Green PCR Master Mix (4367659, Thermo Fisher Scientific), 1 μL of each primer at 10 μmol/μL, and 8 μL of DNA at various concentrations. PCR was performed on the MicroAmp Optical 384-Well Reaction Plates (4326270, Thermo Fisher Scientific) by using the QuantStudio 6 Flex Real-Time PCR System (Thermo Fisher Scientific). After initial denaturation at 95° C. for 10 minutes, cycles of denaturation processes at 95° C. for 15 seconds and 68° C. 30 seconds were repeated.

TABLE 2

| | Normal control group | | Mdx mouse | |
|---|---|---|---|---|
| | Human DNA | | Human DNA | |
| | (100 ng/gDNA) | SEM | (100 ng/gDNA) | SEM |
| Gastrocne | 0.01 | 0 | 2.73 | 0.59 |
| mius | | | | |
| muscle | | | | |
| Heart | 15.00 | 12.14 | 2.29 | 1.09 |
| Liver | 109.49 | 22.66 | 48.82 | 23.57 |
| Lung | 4.43 | 1.71 | 37.42 | 15.09 |
| Spleen | 12.48 | 3.68 | 7.86 | 3.57 |

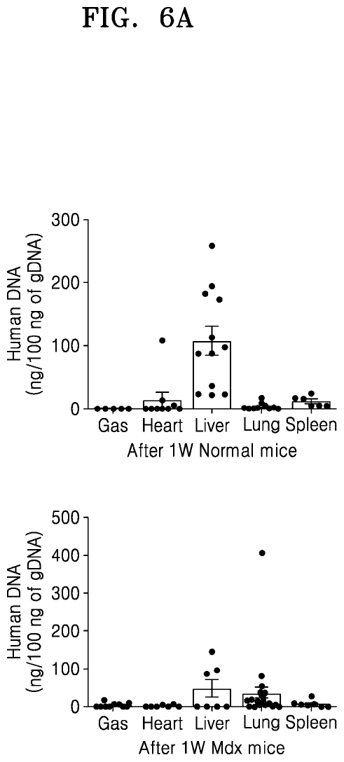

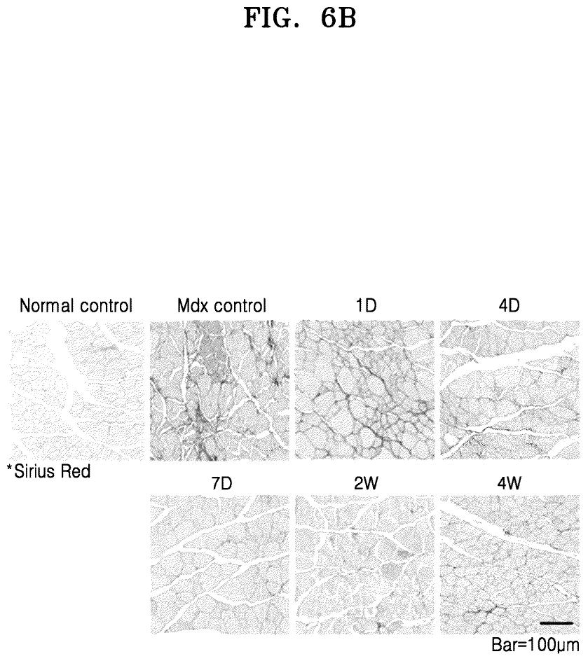

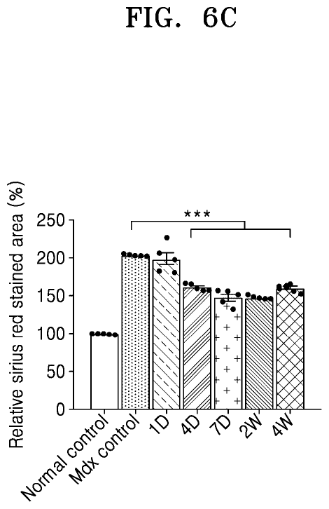

FIG. 6A shows the results of quantifying DNA in organs after intravenous injection of WJ-MSC into a normal control group and mdx mice.

Consequently, as shown in Table 1 and FIG. 6A, it was confirmed that more cells remained in the gastrocnemius muscle of the mdx mice (right) compared to the control group (left). In other words, it was confirmed that the WJ-MCSs intravenously administered to a subject having muscular dystrophy moved to the muscles.

### 4-4. Determination of Duration of WJ-MSC

To confirm the therapeutic effect of mesenchymal stem cells in the treatment of muscular dystrophy, the duration of the anti-fibrotic effect in muscles was confirmed. Specifically, the skeletal muscle of the mdx mice obtained in Example 1-3 was stained with Sirius red in the same manner as in Example 1-3 to observe muscle fibrosis.

FIGS. 6B and 6C show the results of measuring fibrosis over time after intravenous injection of WJ-MSC into mdx mice.

Consequently, as shown in FIGS. 6B and 6C, the fibrosis area was significantly increased in the muscles of the mdx mice as compared to the control group, and was found to decrease continuously for 4 days to 4 weeks after the injection of the WJ-MSCs.

### 4-5. Confirmation of Therapeutic Effect for Fibrosis Caused by Oxidative Stress

The therapeutic effect of mesenchymal stem cells on fibrosis induced by oxidative stress was to be confirmed. Specifically, in the same manner as in Example 4-2, the C2C12 cell line was differentiated into myotubes which were additionally cultured for 24 hours in a growth medium supplemented with 2 mM H₂O₂ (Sigma, MO, USA), so as to induce fibrosis. Afterwards, in a serum-free medium, the myotubes were transfected with 30 nmol/L of human miR-499-5p mimic (GenePharma, Shanghai, China). Also, the fibrosis-induced myotubes in the serum-free medium were treated with 10 μm of galunisertib (LY2157299, S2230, Selleck Chemicals, Houston, TX, USA) for 24 hours. Afterwards, the cells were stained with Sirius red staining in the same manner as in Example 1-3 to confirm the collagen accumulation, and the cells were subjected to immunohistochemistry to confirm the collagen I deposition. Also, to confirm the expression of proteins in the myotubes, Western blot was performed in the same manner as in Example 4-2, except that primary antibodies for MHC (#MAB4470, 1/10,000 dilution; R&D Systems) were additionally used.

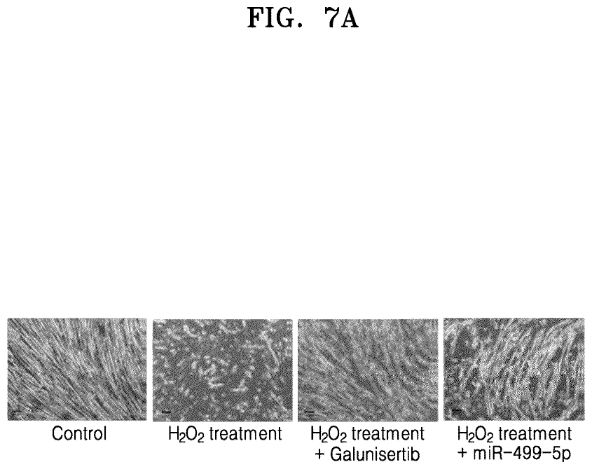

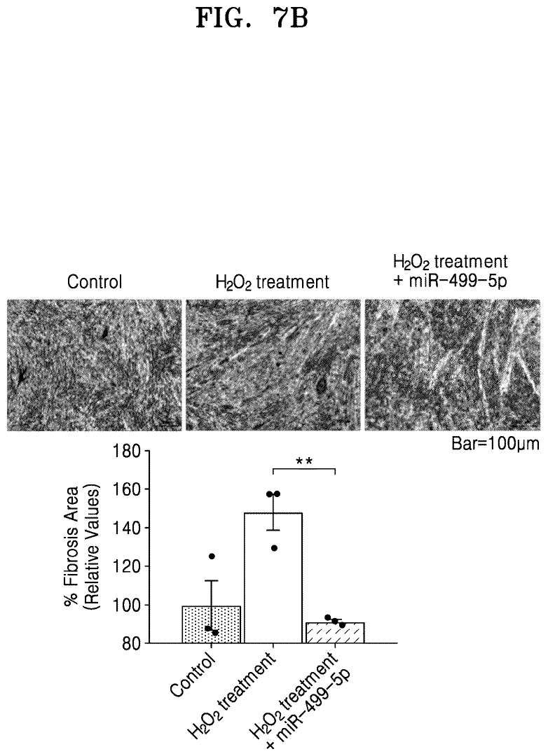

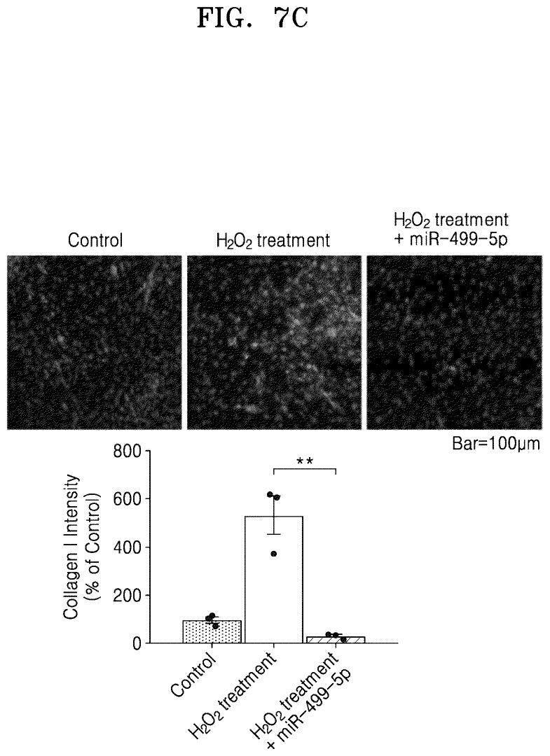

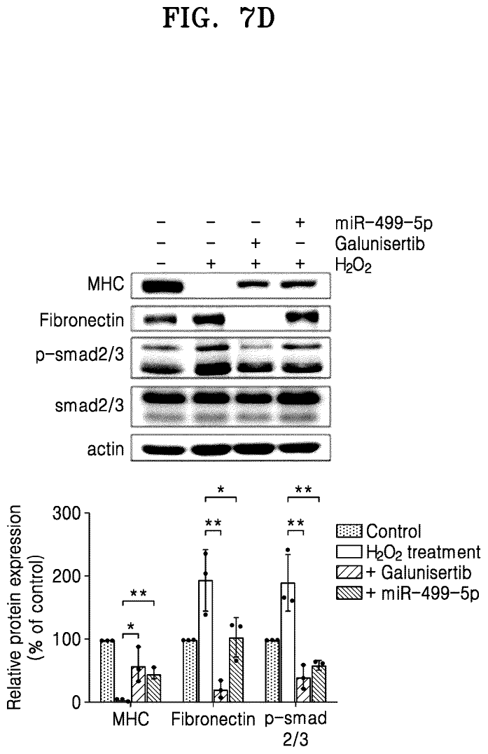

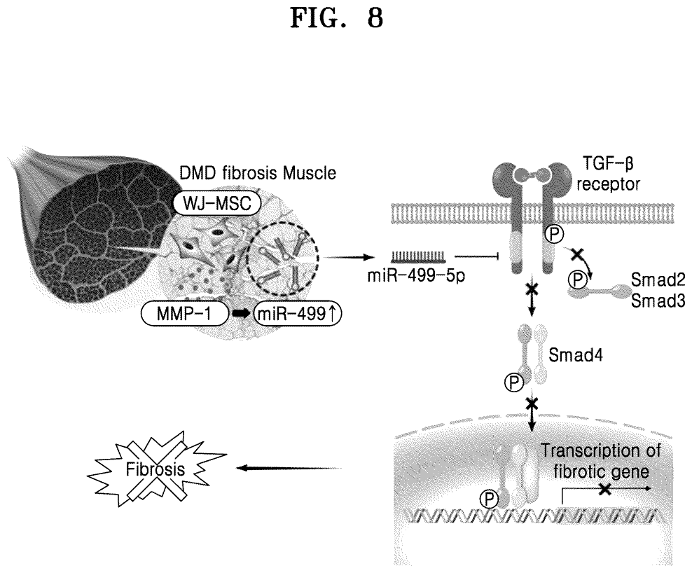

FIG. 7A shows the results of transfection with miR-499-5p or treatment with a TGF-β inhibitor after inducing oxidative stress in mouse myotubes.

Consequently, as shown in FIG. 7A, damage to the myotubes was caused by H₂O₂, but cell apoptosis was not induced. Also, it was confirmed that recovery of damaged myotubes was induced by galunisertib and miR-499-5p.

FIG. 7B shows results confirming collagen accumulation in mouse myotubes transfected with miR-499-5p after inducing oxidative stress in mouse myotubes.

FIG. 7C shows results confirming collagen I accumulation in mouse myotubes transfected with miR-499-5p after inducing oxidative stress in mouse myotubes.

Consequently, as shown in FIGS. 7B and 7C, the accumulation of collagen and deposition of collagen I were significantly increased due to H₂O₂, and was found to recover to a level lower than the control group by the transfection with miR-499-5p. In other words, the miR-499-5p has a therapeutic effect on fibrosis by inhibiting the accumulation of collagen and deposition of collagen I.

FIG. 7D shows the results confirming expression of myosin heavy chain, fibronectin, and p-smad2/3 by inducing oxidative stress in mouse myotubes followed by transfection with miR-499-5p or treatment with a TGF-β inhibitor.

Consequently, as shown in FIG. 7D, the expression of fibronectin and p-smad2/3 was significantly increased due to H₂O₂, and that the expression of fibronectin and p-smad2/3 was found to decrease as compared to the control group. Meanwhile, the expression of MHC decreased due to H₂O₂, but was found to significantly increase by the transfection with miR-499-5p or the treatment with galunisertib. In other words, the anti-fibrosis effect of miR-499-5p can be exhibited by suppressing the TGF-β signaling, and thus can be used for the prevention or treatment of DMD.

The foregoing descriptions are only for illustrating the disclosure, and it will be apparent to a person having ordinary skill in the art to which the present invention pertains that the embodiments disclosed herein can be easily modified into other specific forms without changing the technical spirit or essential features. Therefore, it should be understood that Examples described herein are illustrative in all respects and are not limited.

1. A method for treating Duchenne muscular dystrophy (DMD), comprising administering to a subject a pharmaceutical composition comprising a miR-499-5p activator.

2. The method of claim 1, wherein the miR-499-5p activator comprises Wharton's jelly-derived mesenchymal stem cells (WJ-MSCs).

3. The method of claim 2, wherein the MSCs express MMP-1 or are genetically engineered to overexpress MMP-1 as compared to parent cells.

4. The method of claim 1, wherein the miR-499-5p binds to a TGFβ receptor.

5. The method of claim 1, wherein the pharmaceutical composition is administered intravenously.

6. (canceled)

7. A composition for diagnosing Duchenne muscular dystrophy, the composition comprising an agent that measures an expression level of miR-499-5p.

8. (canceled)

9. A method of providing information for diagnosis of Duchenne muscular dystrophy, the method comprising measuring an expression level of miR-499-5p in a sample of a subject.

10. The method of claim 9, further comprising determining that the subject has Duchenne muscular dystrophy when the expression level of miR-499-5p is decreased as compared to a control group.
